(19) United States
(12) Patent Application Publication
(10) Pub. No.: US 2009/0162458 A1
(43) Pub. Date: Jun. 25, 2009

(54) COMPOSITIONS AND METHODS FOR INDUCING THE EXPRESSION OF HEAT SHOCK PROTEINS

(75) Inventors: Michele Molino, Mississauga (CA); Joseph MacDougall, Mississauga (CA); Phil Apong, Mississauga (CA)

Correspondence Address: TORYS LLP, 79 WELLINGTON STREET WEST, SUITE 3000, BOX 270, TD CENTRE, TORONTO, ON, M5K 1N2, CA

(73) Assignee: H3 FORMULATIONS LTD., Mississauga (CA)

(21) Appl. No.: 11/962,963

(22) Filed: Dec. 21, 2007

(51) Int. Cl. A61K 36/00; C12N 5/00; A61K 31/045

(52) U.S. Cl. 424/769; 435/375; 514/739; 424/725

(57) ABSTRACT

A nutritional composition comprising at least an effective amount of geranylgeranylacetone or derivative of geranygeranylacetone and/or paeoniflorin or derivative of paeoniflorin and an effective amount of Schisandrin B, wherein the ingredients act substantially simultaneously, via differing mechanism, to enhance the expression of heat shock protein in cells, particularly heat shock protein 72 in skeletal muscle. A method of same is also provided.

### FIELD OF THE INVENTION

The present invention relates to a nutritional composition and method for enhancing heat shock protein expression in cells. Specifically, the present invention relates to a composition and method comprising a synergistic combination of at least one substance capable of activating heat shock transcription factors and Schisandrin B, which act substantially simultaneously via differing mechanisms to increase the expression of heat shock proteins in cells, particularly heat shock protein 72 in skeletal muscle, to facilitate increased hypertrophy as a result of exercise.

### BACKGROUND OF THE INVENTION

When a mammalian cell is exposed to a sudden elevation in temperature the expression of most cellular proteins is decreased. However, some proteins, specifically heat shock proteins (HSP), show increased levels of expression when cells are subjected to elevated temperatures and other metabolic stresses. Examples of metabolic stresses which elicit elevated expression of heat shock proteins include: decreased glucose availability; increased intercellular calcium levels; and decreased blood flow.

Heat shock proteins function as molecular chaperones to prevent protein aggregation and facilitate the folding of non-native proteins, particularly new peptides emerging from ribosomes. Molecular chaperones recognize non-native proteins, predominantly via exposed hydrophobic residues, and bind selectively to those proteins to form relatively stable complexes. In these complexes, the protein is protected and able to fold into its native form.

Among the many families of heat shock proteins, HSP72, the stress-inducible protein of the HSP70 family, is one of the best known endogenous factors protecting cells against tissue injury. Research of exercise-induced stress response has shown that exercise results in increased expression of HSP72 mRNA and subsequently in HSP72 protein.

Repetitive, forceful muscular contractions, i.e. physical exercise, cause changes in the expression patterns of genes and proteins. These changes can result in muscle adaptations such as muscle atrophy via muscle protein catabolism or muscle hypertrophy via muscle protein accretion. During hypertrophy, numerous nascent proteins are formed. An increase in the presence of molecular chaperones, such as HSP72, will act to enhance the stability of these nascent proteins until they can fold into their native forms.

In situations of enhanced protein turnover, such as the environment in muscle following exercise, it would be advantageous for an individual to have a means of increasing the stability of rapidly forming proteins in order to reduce the catabolism of these new non-native state proteins.

### SUMMARY OF THE INVENTION

The present invention relates to a nutritional composition and method for enhancing heat shock protein expression in cells. The nutritional composition, comprising an effective amount of at least one substance capable of activating heat shock transcription factors and an effective amount of Schisandrin B acting synergistically, via differing mechanisms, to increase expression of heat shock proteins in cells, particularly heat shock protein 72 in skeletal muscle. Both a composition and a method are provided by the present disclosure.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards a nutritional composition and method for enhancing heat shock protein expression in cells. The nutritional composition, comprising an effective amount of geranylgeranylacetone, paeoniflorin, or a combination thereof, and an effective amount of Schisandrin B functioning synergistically, via differing mechanisms, to increase the expression of heat shock proteins in cells, particularly heat shock protein 72 in skeletal muscle, to facilitate increased hypertrophy as a result of exercise.

A used herein, the term ‘nutritional composition’ includes dietary supplements, diet supplements, nutritional supplements, supplemental compositions and supplemental dietary compositions or those similarly envisioned and termed compositions not belonging to the conventional definition of pharmaceutical interventions as is known in the art. Furthermore, ‘nutritional compositions’, as disclosed herein, belong to a category of compositions having at least one physiological function when administered to a mammal by conventional routes of administration.

Alternatively, formulations and nutritional compositions belonging to the present invention may be considered to be nutraceuticals. As used herein, the term ‘nutraceutical’ is recognized and used in the art to describe a specific chemical compound or combination of compounds found in, organic matter for example, which may prevent, ameliorate or otherwise confer benefits against an undesirable condition. As is known in the art, the term ‘nutraceutical’ is used to refer to any substance that is a food, a part of food, or an extract of food which is suitable for consumption by an individual and provides a physiological benefit which may be medical or health-related. Furthermore, the term has been used to refer to a product isolated, extracted or purified from foods or naturally-derived material suitable for consumption by an individual and usually sold in medicinal forms, such as caplets, tablet, capsules, softgel capsules, gelcaps and the like, not associated with food.

Extracts suitable for use in the present invention may be produced by extraction methods as are known and accepted in the art such as alcoholic extraction, aqueous extractions, carbon dioxide extractions, for example.

As used herein, the term ‘heat shock protein’ is understood to encompass both proteins that are expressly labeled as such as well as other stress proteins, including homologs of such proteins that are expressed in the absence of stressful conditions. Furthermore, as used herein, the term ‘heat shock protein’ is understood to encompass the MRNA species corresponding to expressly labeled heat shock proteins as well as other stress proteins, which are known to be translated into proteins.

Geranylgeranylacetone (GGA)

Geranylgeranylacetone is an acyclic polyisoprenoid that has been used to protect gastric mucosa. GGA has been shown to activate transcription factors, particularly heat shock transcription factor (HSF)-1, which are able to bind to DNA and induce transcription. HSF-1 is normally suppressed since it is typically bound to the C-domain of constitutively active HSP70. GGA is able to bind to the C-domain of the HSP70 thereby causing HSF-1 to dissociate. HSF-1 is now able to undergo trimerization and be translocated to the nucleus, where it binds to the heat shock-responsive element (HSE) in the promoter region of inducible HSP70 (i.e. HSP72) genes.

Recent experiments using cultured mouse skeletal cells, showed that treatment with GGA up-regulated the expression of HSP 72, and increased muscular protein content in a dose-dependent manner. Additionally GGA was shown to facilitate the differentiation of myoblasts into myotubules.

Non-differentiated myoblasts, often referred to as satellite cells, are a small population of quiescent muscle precursor cells that occupy a “satellite” position immediately outside of muscle fibers. They are normally maintained in a quiescent state and become activated to fulfill roles of routine maintenance, repair and hypertrophy. Satellite cells are thought to be muscle-specific stem cells which are capable of producing large numbers of differentiated progeny as well as being capable of self-renewal. Such that satellite cells can fulfill their biological role, they must become activated, proliferate, differentiate and fuse to existing muscle cells. In this way, multinucleate muscle fibers are maintained or increased in size in response to stimuli.

It is herein understood by the inventors that inclusion of geranylgeranylacetone or derivatives of geranylgeranylacetone in a nutritional composition, will act to increase the expression of heat shock proteins, particularly HSP72, via directly activating HSF-1. Enhanced expression of heat shock proteins, particularly HSP72, will act to increase protein accretion via increased stabilization of nascent proteins. The increased expression of chaperone proteins, i.e. HSP72, in working muscle is important in order to stabilize the large number of new proteins being synthesized by working muscle, leading to increased accumulation of contractile protein, i.e. muscle hypertrophy.

Additionally, it is herein understood by the inventors that administration of GGA will have the added benefit of facilitating the differentiation of myoblasts to myofibers. These myofibers fuse with existing muscle cells thereby increasing the size of the muscle cells and ultimately muscle tissue.

As used herein, a serving of the present nutritional composition comprises from about 1 mg to about 300 mg of geranylgeranylacetone or derivatives of geranylgeranylacetone. More preferably, a serving of the present nutritional composition comprises from about 25 mg to about 150 mg of geranylgeranylacetone or derivatives of geranylgeranylacetone. A serving of the present nutritional composition most preferably comprises from about 25 mg to about 75 mg of geranylgeranylacetone or derivatives of geranylgeranylacetone.

Paeoniflorin

Paeoniflorin is a major constituent of peony plants, such as Paeonia lactoflora, P. suffruticosa, P. obovata, and P. veitchii. The roots of peony plants have commonly been used in Chinese medicine to reduce fever and pain, stop bleeding, prevent infection, and as an antispasmodic.

In vitro studies showed that cells treated with paeoniflorin have enhanced levels of expression of heat shock proteins. Paeoniflorin treatment resulted in phosphorylation of HSF-1 allowing HSF-1 to translocate to the nucleus. Inside the nucleus phosphorylated HSF-1 proteins combine to form granules (trimers) which have the ability to bind to the HSE region of inducible heat shock protein genes, thereby inducing transcription of these genes.

It is herein understood by the inventors that inclusion of paeoniflorin or derivatives of paeoniflorin in a nutritional composition, will act to increase the expression of heat shock proteins, particularly HSP72, via directly activating HSF-1. Paeoniflorin or derivatives of paeoniflorin will enhance the expression of HSP by increasing the phosphorylation and DNA-binding ability of HSF-1 thereby facilitating the induction of heat shock proteins. Enhanced expression of heat shock proteins, particularly HSP72, will act to increase protein accretion via increased stabilization of nascent proteins. The increased expression of chaperone proteins, i.e. HSP72, in working muscle is important in order to stabilize the large number of new proteins being synthesized by working muscle, leading to increased accumulation of contractile protein, i.e. muscle hypertrophy.

As used herein, a serving of the present nutritional composition comprises from about 1 mg to about 300 mg of paeoniflorin or derivatives of paeoniflorin. More preferably, a serving of the present nutritional composition comprises from about 1 mg to about 150 mg of paeoniflorin or derivatives of paeoniflorin. A serving of the present nutritional composition most preferably comprises from about 1 mg to about 75 mg of paeoniflorin or derivatives of paeoniflorin.

Schisandrin B

Schisandrin B (Sch B) is a dibenzocyclooctadiene compound that is isolated from Schisandrae chinensis. Sch B has been used to enhance the detoxification of xenobiotics in the liver and assist in liver regeneration. Recent studies have shown that Sch B can protect various organs from free-radical induced damage.

In a study using mice, administration of Sch B was shown to increase the production of HSP70, i.e. HSP72. Treatment with Sch B produces oxidants via cytochrome p-450 metabolism, which act as mild stressors to induce HSP70 (HSP72) production.

It is herein understood by the inventors that inclusion of Schisandrin B in a nutritional composition, will act to increase the production of HSP72, by increasing the production of oxidants from cytochrome P-450 metabolism. Enhanced expression of HSP72, will act to increase protein accretion via increased stabilization of nascent proteins. The increased expression of chaperone proteins, i.e. HSP72, in working muscle is important in order to stabilize the large number of new proteins being synthesized by working muscle, leading to increased accumulation of contractile protein, i.e. muscle hypertrophy.

As used herein, a serving of the present nutritional composition comprises from about 1 mg to about 150 mg of Schisandrin B. More preferably, a serving of the present nutritional composition comprises from about 1 mg to about 75 mg of Schisandrin B. A serving of the present nutritional composition most preferably comprises from about 1 mg to about 25 mg of Schisandrin B.

In embodiments of the present invention, which are set forth in detail in the Examples below, the nutritional composition of the present invention comprises geranylgeranylacetone, paeoniflorin, or a combination thereof, and Schisandrin B. The nutritional composition is provided in any acceptable and suitable oral dosage form as known in the art. Increased expression of heat shock proteins is induced and carried out in an individual by administration of the composition of the present invention.

The nutritional composition of the present invention may be administered in a dosage form having controlled release characteristics, e.g. time-release. Furthermore, the controlled release may be in forms such as a delayed release of active constituents, gradual release of active constituents, or prolonged release of active constituents. Such active constituents release strategies extend the period of bioavailability or target a specific time window for optimal bioavailability. Advantageously the nutritional composition may be administered in the form of a multi-compartment capsule which combines both immediate release and time-release characteristics. Individual components of the nutritional composition may be contained in differential compartments of such a capsule such that the specific components may be released rapidly while others are time-dependently released. Alternatively, a uniform mixture of the various components of the present invention may be divided into both immediate release and time-release compartments to provide a multi-phasic release profile.

According to various embodiments of the present invention, the nutritional supplement may be consumed in any form. For instance, the dosage form of the nutritional supplement may be provided as, e.g., a powder beverage mix, a liquid beverage, a ready-to-eat bar or drink product, a capsule, a liquid capsule, a softgel capsule, a tablet, a caplet, or as a dietary gel. The preferred dosage form of the present invention is as a softgel capsule.

Furthermore, the dosage form of the nutritional supplement may be provided in accordance with customary processing techniques for herbal and nutritional supplements in any of the forms mentioned above. Additionally, the nutritional supplement set forth in the example embodiment herein may contain any appropriate number and type of excipients, as is well known in the art.

The present nutritional composition or those similarly envisioned by one of skill in the art, may be utilized in methods to enhance the expression of heat shock proteins in cells, particularly heat shock protein 72 in skeletal muscle, thereby increasing hypertrophy as a result of exercise.

Although the following examples illustrate the practice of the present invention in various embodiments, the examples should not be construed as limiting the scope of the invention. Other embodiments will be apparent to one of skill in the art from consideration of the specifications and examples.

### EXAMPLES

### Example 1

A nutritional composition comprising the following ingredients per serving are prepared for consumption as three Softgel Capsules, to be taken twice daily:

from about 1 mg to about 300 mg of geranylgeranylacetone, and from about 1 mg to about 150 mg of Schisandrin B.

### Example 2

A nutritional composition comprising the following ingredients per serving are prepared for consumption as four Softgel Capsules, to be taken twice daily:

from about 1 mg to about 300 mg of geranylgeranylacetone, from about 1 mg to about 300 mg of paeoniflorin, and from about 1 mg to about 150 mg of Schisandrin B.

### Example 3

A nutritional composition comprising the following ingredients per serving are prepared for consumption as four Softgel Capsules, to be taken twice daily:

about 50 mg of geranylgeranylacetone, and about 5 mg of Schisandrin B.

### Example 4

A nutritional composition comprising the following ingredients per serving are prepared for consumption as four Softgel Capsules, to be taken twice daily:

about 50 mg of geranylgeranylacetone, about 10 mg of Schisandrin B, and about 100 mg of Ethyl pyruvate.

### Example 5

A nutritional composition comprising the following ingredients per serving are prepared for consumption as four Softgel Capsules, to be taken twice daily:

about 100 mg of geranylgeranylacetone, about 25 mg of Schisandrin B, about 100 mg of Ethyl pyruvate, and about 1 mg of Sulbutiamine.

### Example 6

A nutritional composition comprising the following ingredients per serving are prepared for consumption as three Softgel Capsules, to be taken twice daily:

about 10 mg of geranylgeranylacetone, about 1 mg of Schisandrin B, and about 75 mg of Ethyl pyruvate.

### Example 7

A nutritional composition comprising the following ingredients per serving are prepared for consumption as three Softgel Capsules, to be taken twice daily:

about 50 mg of geranylgeranylacetone, about 1 mg of paeoniflorin, and about 1 mg of Schisandrin B.

### Extensions and Alternatives

In the foregoing specification, the invention has been described with a specific embodiment thereof; however, it will be evident that various modifications and changes may be made thereto without departing from the broader spirit and scope of the invention.

What is claimed:

1. A nutritional composition for enhancing heat sock protein expression in cells, comprising;
from about 1 mg to about 300 mg of at least one substance capable of activating heat shock transcription factors; and
from about 1 mg to about 150 mg of Schisandrin B or derivatives of Schisandrin B.

2. The composition of claim 1, wherein the at least one substance capable of activating heat shock transcription factors is selected from the group consisting of geranylgeranylacetone or derivatives of geranylgeranylacetone, and paeoniflorin or derivatives of paeoniflorin.

3. The composition of claim 1, wherein the at least one substance capable of activating heat shock transcription factors is geranylgeranylacetone.

4. The composition of claim 1, wherein the at least one substance capable of activating heat shock transcription factors is a combination of geranylgeranylacetone and paeoniflorin.

5. The composition of claim 3, wherein the amount of the geranylgeranylacetone is about 50 mg.

6. The composition of claim 4, wherein the amount of the geranylgeranylacetone is about 50 mg, and the amount of paeoniflorin is about 1 mg.

7. The composition of claim 1, where in the amount of the Schisandrin B is about 1 mg.

8. The composition of claim 1, wherein the heat shock protein is heat shock protein 72 and the cell is skeletal muscle.

9. The composition of claim 1, wherein enhanced expression of heat shock protein in cells facilitates increased hypertrophy as a result of exercise.

10. A method of enhancing the expression of heat shock protein in cells, comprising the step of administering to a mammal a composition comprising;
from about 1 mg to about 300 mg of at least one substance capable of activating heat shock transcription factors; and
from about 1 mg to about 150 mg of Schisandrin B or derivatives of Schisandrin B.

11. The method of claim 10, wherein the at least one substance capable of activating heat shock transcription factors is selected from the group consisting of geranylgeranylacetone or derivatives of geranylgeranylacetone, and paeoniflorin or derivatives of paeoniflorin.

12. The method of claim 10, wherein the at least one substance capable of activating heat shock transcription factors is geranylgeranylacetone.

13. The method of claim 10, wherein the at least one substance capable of activating heat shock transcription factors is a combination of geranylgeranylacetone and paeoniflorin.

14. The method of claim 12, wherein the amount of the geranylgeranylacetone is about 50 mg.

15. The method of claim 13, wherein the amount of the geranylgeranylacetone is about 50 mg, and the amount of paeoniflorin is about 1 mg.

16. The method of claim 10, where in the amount of the Schisandrin B is about 1 mg.

17. The method of claim 10, wherein the heat shock protein is heat shock protein 72 and the cell is skeletal muscle.

18. The method of claim 10, wherein enhanced expression of heat shock protein in cells facilitates increased hypertrophy as a result of exercise.

19. A nutritional composition for enhancing heat sock protein expression in cells, comprising:
about 50 mg of geranylgeranylacetone;
about 1 mg of paeoniflorin; and
about 1 mg of Schisandrin B.
